(19) United States
(12) Patent Application Publication
(10) Pub. No.: US 2011/0155135 A1
(43) Pub. Date: Jun. 30, 2011

(54) ISOLATION VALVE FOR A CLOSED SUCTION DEVICE

(75) Inventor: SHENG-YU CHIU, HOU LI HSIANG (TW)

(73) Assignee: VITALTEC CORPORATION, Hou Li Hsiang (TW)

(21) Appl. No.: 12/650,668

(22) Filed: Dec. 31, 2009

(51) Int. Cl. A61M 16/20; A61M 1/00

(52) U.S. Cl. 128/205.24

(57) ABSTRACT

An isolation valve for a closed suction device has a body and a valve. The body has a communicating channel, a patient end connector, a breathing circuit connector and a suction tube connector. The communicating channel has a chamber and an opening. The patient end connector, the breathing circuit connector and the suction tube connector are radially formed on and protrude from the external surface of the communicating channel and communicate with the chamber of the communicating channel. The valve is rotatably connected to the body and has an isolation pipe and a control button. The isolation pipe is rotatably mounted in the chamber of the communicating channel via the opening and has a communicating hole, an inlet hole and a sucking hole. The control button is formed on the isolation pipe above the top of the communicating channel

### BACKGROUND OF THE INVENTION

1. Field of the Invention

The present invention relates to an isolation valve, and more particularly relates to an isolation valve for a closed suction device that provides a simplified design in lightweight to reduce the impact on patient, is operated easily and reduces manufacturing costs.

2. Description of Related Art

A closed suction device is used to draw secretion from a patient with a drawing force provided by a hospital vacuum source without moving a breathing circuit connector. Furthermore, the closed suction device is used to perform secretion suction on a patient while providing ventilation by the breathing circuit connector simultaneously. Consequently, this can reduce chances of negatively influencing patient safety.

Drawing secretion from a patient, a connecting device is built up on the conventional closed suction device to connect the patient end. The invention is to design an isolation valve in the connecting device to open or close the tubes between the closed suction device. Based on the isolation valve, the patient could maintain PEEP level and waste liquid is prevented from back-flowing to the patient during rinsing, which could prevent the patient's respiratory passage from infection during secretion suction. Furthermore, cross infection between the patients and medical personnel, patients and patients in a hospital can be avoided further. However, the isolation valve of the connecting device on an available closed suction device has complicated structure and cannot be operated conveniently. Also manufacturing costs of the isolation valve for a closed suction device are increased.

Therefore, the present invention provides an isolation valve to mitigate or obviate the aforementioned problems.

### SUMMARY OF THE INVENTION

The main objective of the present invention is to provide an isolation valve for a closed suction device that can provide a simplified design, be operated easily and reduce manufacturing costs.

The isolation valve for a closed suction device in accordance with the present invention has a body and a valve. The body has a communicating channel, a patient end connector, a breathing circuit connector and a suction tube connector. The communicating channel has a chamber and an opening. The patient end connector, the breathing circuit connector and the suction tube connector are radially formed on and protrude from an external surface of the communicating channel and communicate with the chamber of the communicating channel. The valve is rotatably connected to the body and has an isolation pipe and a control button. The isolation pipe is rotatably mounted in the chamber of the communicating channel via the opening and has a communicating hole, an inlet hole and a sucking hole. The control button is formed on the isolation pipe above the top of the communicating channel.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an isolation valve for a closed suction device in accordance with the present invention;

FIG. 2 is an exploded perspective of the isolation valve in FIG. 1;

FIG. 3 is an operational side view in partial section of the isolation valve in FIG. 1 connected to a suction device; and

FIG. 4 is another operational side view in partial section of the isolation valve in FIG. 1.

### DETAILED DESCRIPTION OF THE PREFERRED EMBODIMENT

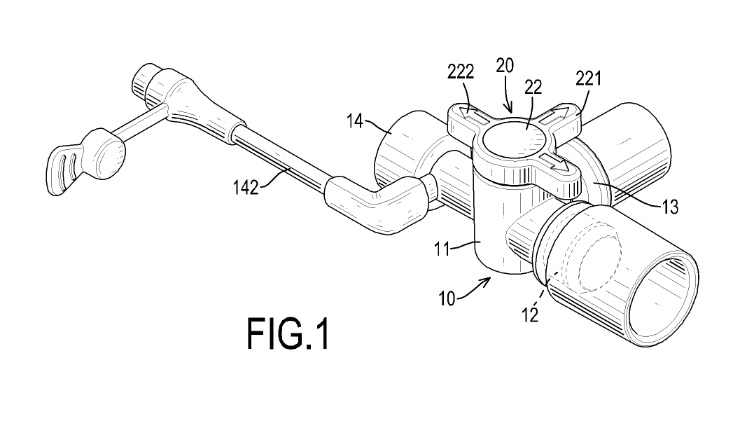

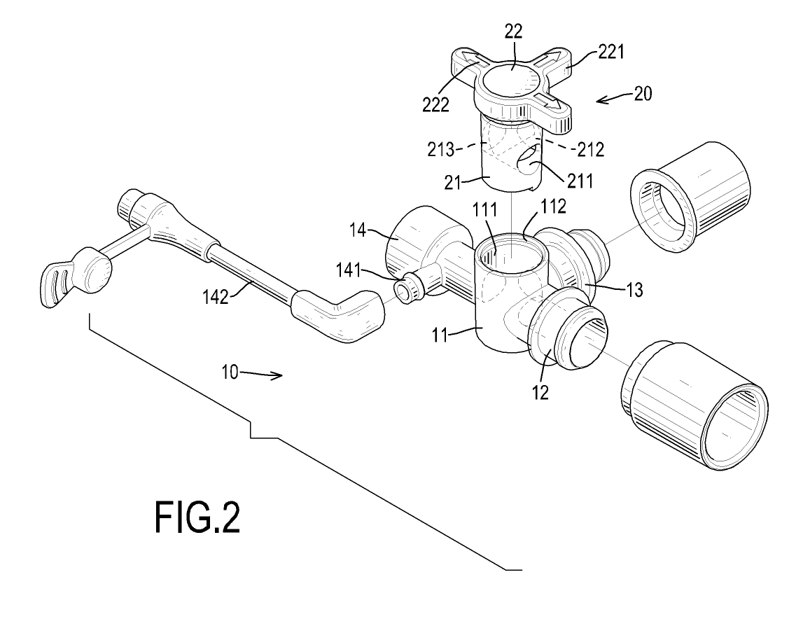

With reference to FIGS. 1 to 3, an isolation valve for a closed suction device in accordance with the present invention has a body (10) and a valve (20).

The body (10) has a communicating channel (11), a patient end connector (12), a breathing circuit connector (13) and a suction tube connector (14).

The communicating channel (11) is hollow and has a top, a bottom, an external surface, a chamber (111) and an opening (112). The chamber (111) is formed in the communicating channel (11) between the top and the bottom of the communicating channel (11). The opening (112) is formed through the top of the communicating channel (11) and communicates with the chamber (111).

The patient end connector (12) is radially formed on and protrudes from the external surface of the communicating channel (11), communicates with the chamber (111) of the communicating channel (11) and is connected to an airway device that is connected to a patient.

The breathing circuit connector (13) is radially formed on and protrudes from the external surface of the communicating channel (11), communicates with the chamber (111) of the communicating channel (11) and is connected to a respiratory system. Preferably, the breathing circuit connector (13) is perpendicular to the patient end connector (12).

The suction tube connector (14) is radially formed on and protrudes from the external surface of the communicating channel (11), communicates with the chamber (111) of the communicating channel (11) and is connected to the suction tube (30) and has an external surface, a connecting port (141), an irrigation tube (142), a secretion removing membrane (143) and an irrigation chamber (144).

The connecting port (141) is formed on and protrudes from the external surface of the suction tube connector (14) and has a free end. The irrigation tube (142) is detachably mounted around the free end of the connecting port (141) to inject liquid into the irrigation chamber (144) of the suction tube connector (14) to irrigate the suction tube (30). The secretion removing membrane (143) is mounted in the suction tube connector (14) near the connecting port (142). The irrigation chamber (144) is formed in the suction tube connector (14) between the communicating channel (11), the connecting port (141) and the secretion removing membrane (143). Preferably, the suction tube connector (14) aligns with the patient end connector (12) and is perpendicular to the breathing circuit connector (13).

The valve (20) is rotatably connected to the body (10) and has an isolation pipe (21) and a control button (22).

The isolation pipe (21) is rotatably mounted in the chamber (111) of the communicating channel (11) via the opening (112) and has a top, an external surface, a communicating hole (211), an inlet hole (212) and a sucking hole (213). The communicating hole (211) is radially formed through the external surface of the isolation pipe (21) and communicates with the patient end connector (12). The inlet hole (212) is radially formed through the external surface of the isolation pipe (21) and communicates with the breathing circuit connector (13). The sucking hole (213) is radially formed through the external surface of the isolation pipe (21) and communicates with the suction tube connector (14).

The control button (22) is formed on the top of the isolation pipe (21) above the top of the communicating channel (11) and has a periphery, multiple stems (221) and multiple direction marks (222). The stems (221) are formed on and protrude from the periphery of the control button (22) and each stem (221) has a top face. In a preferred embodiment, the control button (22) has three stems (221) respectively aligning with the patient end connector (12), the breathing circuit connector (13) and the suction tube connector (14). The direction marks (222) are respectively formed on the top faces of the stems (221) to provide a direction effect.

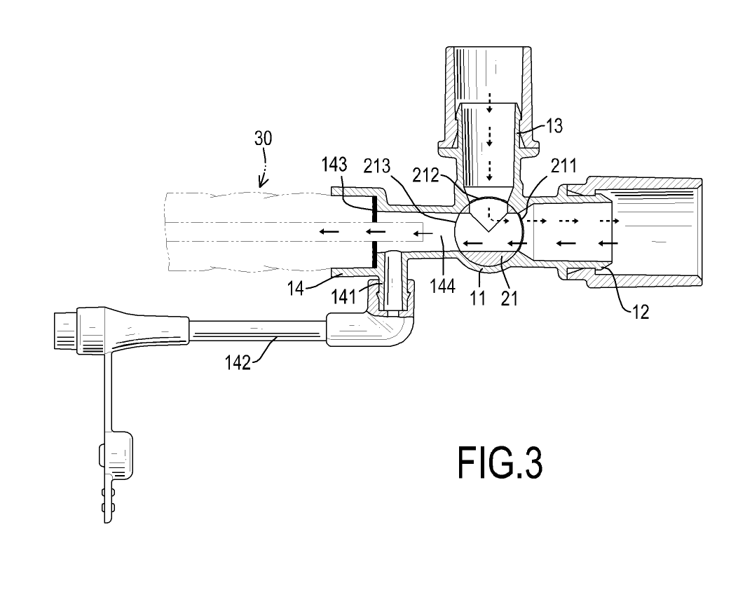

With reference to FIG. 3, when the isolation valve in accordance with the present invention is used to draw secretion from a patient, a suction tube (30) with a protection sleeve is inserted into the suction tube connector (14) abutting the secretion removing membrane (143) and the control button (22) of the valve (20) is rotated to respectively align the communicating hole (211), the inlet hole (212) and the sucking hole (213) with the patient end connector (12), the breathing circuit connector (13) and the suction tube connector (14) of the body (10). Then, a drawing force is provided by a vacuum source that is connected to the suction tube (30) opposite to the suction tube connector (14) to draw secretion from a patient. Consequently, the secretion or sputum of the patient can be drawn from the patient end connector (12) to the suction tube (30) via the communicating hole (211), the sucking hole (213) and the suction tube connector (14). In addition, the dephlogisticated air can be supplied to the patient from the breathing circuit connector (13) to the patient end connector (12) via the inlet hole (212) and the communicating hole (211) during suction.

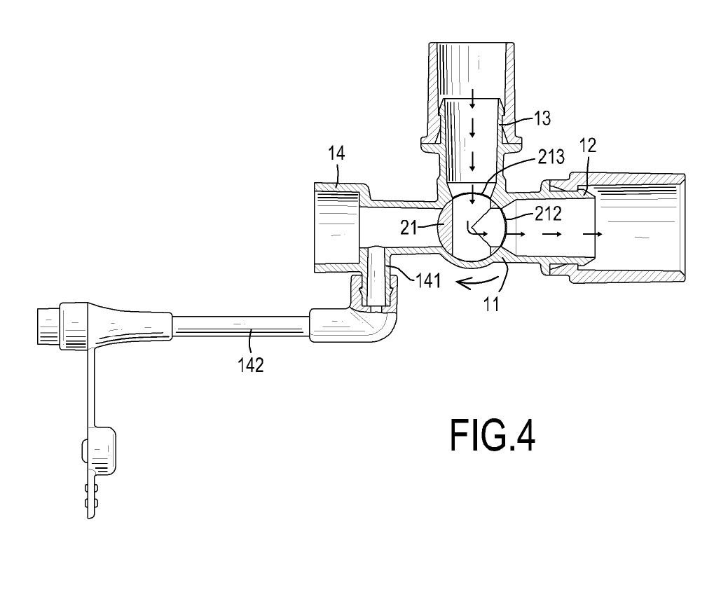

With reference to FIG. 4, after the secretion or the sputum of the patient is drawn into the suction tube (30), the control button (22) of the valve (20) is rotated relative to the communicating channel (11) of the body (10) to make the sucking hole (213) and the inlet hole (212) of the isolation pipe (21) respectively communicate with the breathing circuit connector (13) and the patient end connector (12). When the control button (22) is rotated to make the sucking hole (213) and the inlet hole (212) of the isolation pipe (21) respectively communicate with the breathing circuit connector (13) and the patient end connector (12), the suction tube connector (14) is closed and kept from communicating with the breathing circuit connector (13) and the patient end connector (12). Consequently, cross infection between patients and medical personnel in a hospital can be avoided.

Furthermore, the secretion or the sputum remained in the suction tube connector (14) can be irrigated by liquid injecting into the irrigation chamber (144) via the irrigation tube (142) and the connecting port (141), and the waste liquid in the suction tube connector (14) will not back-flowing to the patient during the rinsing process.

The isolation valve for a closed suction device as described has the following advantages.

1. The design of the isolation valve for a closed suction device is simplified and in lightweight to reduce the impact on patient, and also will reduce the cost for manufacturing the isolation valve.

2. When the isolation valve in accordance with the present invention is used to draw secretion from a patient, the user only needs to rotate the control button (22) of the valve (20) to open or close the pipes. Therefore, the operation of the isolation valve for a closed suction device is easy.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An isolation valve for a closed suction device having
a body having
a communicating channel being hollow and having
a top;
a bottom;
an external surface;
a chamber formed in the communicating channel between the top and the bottom of the communicating channel; and
an opening formed through the top of the communicating channel and communicating with the chamber;
a patient end connector radially formed on and protruding from the external surface of the communicating channel and communicating with the chamber of the communicating channel—;
a breathing circuit connector radially formed on and protruding from the external surface of the communicating channel and communicating with the chamber of the communicating channel—; and
a suction tube connector radially formed on and protruding from the external surface of the communicating channel and communicating with the chamber of the communicating channel—; and
a valve rotatably connected to the body and having
an isolation pipe rotatably mounted in the chamber of the communicating channel via the opening and having
a top;
an external surface;
a communicating hole radially formed through the external surface of the isolation pipe and communicating with the patient end connector;
an inlet hole radially formed through the external surface of the isolation pipe and communicating with the breathing circuit connector; and
a sucking hole radially formed through the external surface of the isolation pipe and communicating with the suction tube connector; and
a control button formed on the top of the isolation pipe above the top of the communicating channel—and having
a periphery; and
multiple stems formed on and protruding from the periphery of the control button.

2. The isolation valve as claimed in claim 1, wherein
the breathing circuit connector is perpendicular to the patient end connector; and
the suction tube connector aligns with the patient end connector and is perpendicular to the breathing circuit connector.

3. The isolation valve as claimed in claim 2, wherein the suction tube connector has
an external surface;
a connecting port formed on and protruding from the external surface of the suction tube connector and having a free end;
an irrigation tube detachably mounted around the free end of the connecting port for injecting liquid into the suction tube connector to rinse the suction tube via the irrigation tube;
a secretion removing membrane mounted in the suction tub connector near the connecting port; and
an irrigation chamber formed in the suction tube connector between the communicating channel, the connecting port and the secretion removing membrane.

4. The isolation valve as claimed in claim 3, wherein the control button has three stems respectively aligning with the patient end connector, the breathing circuit connector and the suction tube connector.

5. The isolation valve as claimed in claim 4, wherein
each stem has a top face; and
the control button has multiple direction marks respectively formed on the top faces of the stems to provide a direction effect.

6. The isolation valve as claimed in claim 1, wherein the suction tube connector has
an external surface;
a connecting port formed on and protruding from the external surface of the suction tube connector and having a free end;
an irrigation tube detachably mounted around the free end of the connecting port for injecting liquid into the irrigation chamber of the suction tube connector to rinse the suction tube via the irrigation tube;
a secretion removing membrane mounted in the suction tube connector near the connecting port; and
an irrigation chamber formed in the suction tube connector between the communicating channel, the connecting port and the secretion removing membrane.

7. The isolation valve as claimed in claim 1, wherein the control button has three stems respectively aligning with the patient end connector, the breathing circuit connector and the suction tube connector.

8. The isolation valve as claimed in claim 7, wherein
each stem has a top face; and
the control button has multiple direction marks respectively formed on the top faces of the stems to provide a direction effect.
